(19) United States
(12) Patent Application Publication
(10) Pub. No.: US 2012/0021405 A1
(43) Pub. Date: Jan. 26, 2012

(54) Single Chain Antibody for the Detection of Noroviruses

(75) Inventors: Timothy Palzkill, Houston, TX (US); Wanzhi Huang, Houston, TX (US); Mary K. Estes, Houston, TX (US)

(73) Assignee: Baylor College of Medicine, Houston, TX (US)

(21) Appl. No.: 13/146,155

(22) PCT Filed: Jan. 26, 2010
(86) PCT No.: PCT/US2010/022072

§ 371 Date: Oct. 6, 2011

Related U.S. Application Data
(60) Provisional application No. 61/147,247, filed on Jan. 26, 2009.

(51) Int. Cl. C12Q 1/70; C12N 1/19; C12N 5/10; C12N 1/21; C07K 16/10; C12N 15/63

(52) U.S. Cl. 435/5; 530/326; 530/329; 530/328; 530/324; 435/320.1; 435/328; 435/252.33; 435/254.2

(57) ABSTRACT

The present invention concerns compositions and methods for detecting Norovirus or Norovirus particles. In particular, the present invention encompasses antibodies for detecting Norovirus or Norovirus particles, including, for example, monoclonal antibodies that have broad specificity of binding to various genogroups of norovirus.

### CROSS-REFERENCE TO RELATED APPLICATIONS

This application claims priority to U.S. Provisional Application Ser. No. 61/147,247, filed on Jan. 26, 2009, which is incorporated by reference herein in its entirety.

### STATEMENT REGARDING FEDERALLY SPONSORED RESEARCH OR DEVELOPMENT

This invention was made with government support under P01 AI 057788 awarded by the National Institutes of Health. The government has certain rights in the invention.

### TECHNICAL FIELD

The present invention concerns at least the fields of cell biology, molecular biology, immunology, and medicine.

### BACKGROUND OF THE INVENTION

Noroviruses (NoV) are recognized as a leading cause of acute gastroenteritis (Beuret et al., 2003; Atmar and Estes, 2006; Tsang et al., 2008). They are associated with large outbreaks involving hospitals, nursing homes, cruise ships, schools and other institutional settings. The infectious dose of virus is thought to be <100 virions and transmission of the virus can be food-borne, person-to-person or from environmental sources (Parashar and Monroe, 2001; Atmar and Estes, 2006; Lee et al., 2007; Teunis et al., 2008).

NoVs are small, round RNA viruses that belong to the family Caliciviridae. They contain a single-stranded, positive sense genome that encodes three open reading frames (ORFs). ORF1 encodes non-structural proteins such as the polymerase and protease while ORF2 encodes the major capsid protein (VP1) and ORF3 encodes a minor structural protein (VP2) (Jiang et al., 1990). Expression of ORF2 from insect cells results in the self-assembly of VP1 into virus-like particles (VLPs) which are antigenically and morphologically similar to native virions (Jiang et al., 1992; Green et al., 1993). Structural studies of VLPs and VP1 indicate the major capsid protein can be divided into three domains with the shell (S) domain located at the interior and the protruding domains P1 and P2 progressively more exposed on the surface (Prasad et al., 1999). VLPs bind to carbohydrates from human blood group antigens (HBGA) that are found on the surface of intestinal epithelial cells and these sites are thought to serve as the receptor for virus (Hutson et al., 2003; Hutson et al., 2004). The X-ray crystal structure of the P-domain of Norwalk virus in complex with A- and H-type HBGAs indicates the P2 domain contains the binding site for carbohydrate (Bu et al., 2008; Choi et al., 2008). Structural studies further indicate that the position of the HBGA binding site on the P2 domain varies between genogroups of NoV (Cao et al., 2007; Choi et al., 2008).

NoVs are a genetically diverse group of viruses that have been classified into five genogroups (I-V) based on the major capsid sequence (Zheng et al., 2006). The human noroviruses include genogroups I, II and IV and these have been further subdivided into at least 8 GI and 17 GII groups (Zheng et al., 2006). A large amount of amino acid sequence diversity occurs in VP1, particularly in the P2 protruding domain (Chen et al., 2004). The diversity of NoV strains creates a challenge in the development of diagnostic assays that can be used to broadly detect NoVs. Three methods that have been used include electron microscopy (EM), RT-PCR or, more recently, real time RT-PCR, as well as enzyme linked immunoabsorbant assays (ELISA) (Ando et al., 1995; Jiang et al., 2000; Atmar and Estes, 2006; Wolf et al., 2007). Electron microscopy can be used to directly detect virions in stool samples; however, the method is work intensive and is less sensitive than molecular methods (Richards et al., 2003). RT-PCR is the most widely used method of detection and involves the use of virus-specific primers that are complementary to conserved regions in the genome (Atmar and Estes, 2001). The sequence diversity of NoV prohibits the use of a single primer pair for broad detection, however the inclusion of two primer pairs allows detection of >90% of GI and GII viruses (Blanton et al., 2006).

The detection of viral antigens in stool by ELISA has also been used as a tool to diagnose NoV infections (Jiang et al., 2000). ELISA-based methods have the advantage of ease of use and do not require specialized equipment. The difficulty with this approach has been the specificity of the assay. Because of the extensive diversity of the P domain of VP1, antibodies that recognize viral surface antigens are quite specific for genogroups or even subgroups of NoV (Jiang et al., 1995). For example, hyperimmune sera raised against genogroup I or genogroup II VLPs were found to react specifically with GI or GII samples, respectively (Jiang et al., 1995; Atmar and Estes, 2001). Monoclonal antibodies have been developed, however, that are more broadly reactive within a genogroup and these have been used for diagnostic assays (Parker et al., 2005). These ELISA-based diagnostic assays exhibit modest sensitivity (38%, Dako; 36%, Ridascreen) but high specificity (96%, Dako; 88%, Ridascreen)(de Bruin et al., 2006). Therefore, there is a need for the development of antibodies that can bind tightly to a broad range of GI and GII samples that could be used to enhance the sensitivity of ELISA-based diagnostic assays.

The monoclonal antibodies commonly used for norovirus detection were obtained from mice following oral or intraperitoneal inoculation and with standard hybridoma procedures (Hardy et al., 1996; Kitamoto et al., 2002; Parker et al., 2005). Another approach to obtain monoclonal antibodies is to use phage display to isolate antibodies of interest from large combinatorial libraries (Sidhu and Fellhouse, 2006; Michnick and Sidhu, 2008). Synthetic antibody libraries consist of a single framework with the molecular diversity created in antigen binding sites by site directed mutagenesis (Sidhu and Fellhouse, 2006). In the present invention, norovirus VLPs were used as targets for biopanning of a monoclonal human single-chain antibody (scFv) library by phage display to identify antibody fragments that bind to GI and GII norovirus VLPs. Several antibodies that target noroviruses were obtained and characterized. The antibodies are useful at least as detection reagents for ELISA-based diagnostic assays.

### BRIEF SUMMARY OF THE INVENTION

The present invention is directed to at least one system, method and/or composition for the detection of Noroviruses. In specific embodiments, the invention concerns identification of human single-chain antibodies that target Norovirus virus-like particles, although in certain embodiments they target Norovirus virus particles. In particular cases, the antibodies identified by the present invention and/or encompassed thereby are employed to detect Norovirus infection in mammalian individuals, including humans.

Certain embodiments of the present invention include specific sequences that are employed in the antigen-detecting regions. In particular embodiments, one or more of the sequences are located in a complementarity determining region (CDR) of an antibody of the invention. The skilled artisan recognizes that the CDR is a relatively short amino acid sequence that determines the specificity and makes contact with a specific ligand, in this case Norovirus virus-like particles or Norovirus virus particles.

In some embodiments, there is a peptide, for example an isolated peptide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

In particular embodiments there is an antibody, for example an isolated antibody, comprising sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and a combination thereof. In a specific embodiment, the antibody is further defined as a single chain antibody.

In some embodiments, there is an antibody, for example an isolated antibody, comprising: 1) an amino acid segment comprising sequence selected from the group consisting of SEQ ID NO:5, SEQ ID NO:9, and SEQ ID NO:10; 2) an amino acid segment comprising sequence selected from the group consisting of SEQ ID NO:6, SEQ ID NO:11, and SEQ ID NO:12; 3) an amino acid segment comprising sequence selected from the group consisting of SEQ ID NO:7, SEQ ID NO:13, SEQ ID NO:14, SEQ ID NO:15, SEQ ID NO:16, and SEQ ID NO:17; 4) an amino acid segment comprising sequence selected from the group consisting of SEQ ID NO:8, SEQ ID NO:18, SEQ ID NO:19, SEQ ID NO:20, SEQ ID NO:21, SEQ ID NO:22, and SEQ ID NO:23; and 5) a combination thereof. In a specific embodiment, the antibody is further defined as a single chain antibody.

Embodiments of the invention include one or more kits comprising an antibody of the invention and, in some cases, includes other reagents suitable for detecting and/or treating Norovirus infection.

In certain embodiments, there is a method of detecting genogroup II-4 Noroviruses in an individual having or suspected of having Norovirus infection, comprising the step of obtaining a sample from an individual and subjecting the sample to an antibody of the invention. In specific embodiments, the antibody is labeled.

In certain embodiments of the invention, there is a method of detecting Norovirus genogroups I or II in an individual having or suspected of having Norovirus infection, comprising the step of obtaining a sample from an individual and subjecting the sample to an antibody of the invention. In a specific embodiment, the antibody in the subjecting step is further defined as a detection antibody in a sandwich ELISA method. In another specific embodiment, the antibody is labeled.

In certain embodiments of the invention, there is a method of testing for Norovirus infection in an individual suspected of having a Norovirus infection or having been exposed to Norovirus, comprising the steps of obtaining a stool sample from the individual, wherein the individual has nausea, abdominal pain, abdominal cramps, and/or diarrhea; and subjecting the sample to an antibody of the invention. In a specific embodiment, the antibody is labeled.

In some embodiments of the invention, there is a method of testing for Norovirus infection in an individual suspected of having a Norovirus infection, comprising the steps of obtaining a stool sample from the individual, wherein the individual has nausea, abdominal pain, abdominal cramps, and/or diarrhea; and subjecting the sample to an antibody of the invention. In a specific embodiment, the antibody is labeled.

In some embodiments of the invention, there is an expression construct that encodes a peptide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

In particular embodiments of the invention, there is an isolated cell housing an expression construct that encodes a peptide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23. In specific embodiments, the cell is an E. coli, yeast, mammalian, or insect cell, for example.

In certain embodiments of the invention, there is protein overexpression of the entire antibody in an E. coli, yeast, mammalian, or insect cell, for example.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1. Amino acid sequences of scFv clones obtained after three rounds of binding enrichment on HOV VLPs followed by trypsin elution and ELISA screening for clones that also bound to NV VLPs. The complementarity determining regions (CDR) positions that are randomized in the Tomlinson J library are highlighted with underlining. The asterisk indicates the amber TAG stop codon which is suppressed to glutamine in the E. coli strain used to propagates phages.

FIG. 2. Amino acid sequences of scFv clones obtained after three rounds of binding enrichment on HOV VLPs followed by carbohydrate elution ELISA screening. The CDR positions that are randomized in the Tomlinson J library are highlighted with underlining. The asterisk indicates the amber TAG stop codon which is suppressed to glutamine in the E. coli strain used to propagates phages.

FIG. 3. Surface plasmon resonance sensogram showing binding of HJT-R3-A9 scFv antibody (16 nM) to Houston (HOV) and Norwalk (NV) VLPs that were immobilized to the surface of the SPR chip. Binding of HJT-R3-A9 antibody to immobilized TEM-1 β-lactamase is included as a control indicating the antibody binding is specific for VLPs. The arrow indicates the point at which the solution flowing over the chip contained buffer only.

FIG. 4. ELISA measurement of binding of purified, soluble HJT-R3-A9 scFv to immobilized HOV and NV VLPs. TEM-1 β-lactamase was also immobilized in ELISA wells as a negative control. The VLPs and TEM-1 β-lactamase were coated into wells at concentrations of 5 μg/ml (open bars), 10 μg/ml, (gray bars), 20 μg/ml (striped bars) and 40 μg/ml (black bars).

FIG. 5. Immunoblot to test binding of HJT-R3-A9 antibody to HOV, NV and CT303 VLPs and GST-GI-P-domain protein. Lanes 1,4,12,-MW MARKER; 2-HOV VLP; 3-NV VLP; 4-blank; 5-GST-P-domain; 6-GST; 7-GST-P-domain (not boiled); 8-GST(not boiled); 9-CT303 VLP (S-domain); 10-CT303 VLP (not boiled); 11-TEM-1 β-lactamase.

FIG. 6. Phage ELISA of HJT-R3-A9 and HJL-R3 phages binding to HOV VLPs (filled bars), GST-HOV P domain (open bars), GST alone (striped bars) and E. coli maltose binding protein (gray bars). Phage displaying scFv antibodies were added to each immobilized protein, washed, and bound phage were detected with anti-M13 antibody. The names of the scFv phages used are listed under the X-axis.

FIG. 7. ELISA to measure capture of VLPs of various norovirus GII subtypes by scFv antibody. Purified scFv of HJT-R3-A9 (striped bars), HJL-R3-B4 (gray bars), HJL-R3-D11 (open bars) and HJL-R3-F11 (filled bars) were coated into ELISA wells and VLPs from GII subtypes were added. VLPs that were bound by immobilized by scFv were detected with anti-HOV rabbit polyclonal antibody.

FIG. 8. Test of scFvs as detection antibodies for norovirus VLPs. ELISA wells coated with NS-14 monoclonal antibody that detects genogroup II VLPs. VLPs from several different genogroup II subgroups including HOV (GII-4) were added and allowed to bind the immobilized NS-14. Maltose binding protein was added rather than a VLP as a negative control. The HJT-R3-A9 and HJL-R3-B4, D11, F11 scFv proteins were added, washed and detected with an anti-His-tag antibody. Black bars, HJT-R3-A9; gray bars, HJL-R3-B4; striped bars, HJL-R3-D11; white bars, HJL-R3-F11.

FIG. 9. Detection of norovirus in clinical samples using scFv HJL-R3-B4. ELISA wells were coated with NS-14 antibody. 10% stool suspensions of norovirus GII-4 positive and negative samples were added as indicated. A positive control sample of purified HOV VLP (1.6 μg/well) and negative control of PBS alone were also added to separate wells as indicated.

### DETAILED DESCRIPTION OF THE INVENTION

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references, for example.

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of or “consist of one or more sequences of the invention, for example. Some embodiments of the invention may consist of or consist essentially of one or more elements, method steps, and/or methods of the invention. It is contemplated that any method or composition or part therein described herein can be implemented with respect to any other method or composition described herein.

### I. General Embodiments of the Invention

Norovirus infections are a common source of gastroenteritis. New methods to rapidly diagnose norovirus infections are needed in the art. In particular embodiments, the present invention describes new monoclonal antibodies that have broad specificity of binding to various genogroups of norovirus. A human scFv phage display library was used to identify an exemplary antibody, HJT-R3-A9, which binds tightly to both genogroup I and II norovirus VLPs. Studies to determine the binding site on VLPs indicated the antibody binds to the S-domain. In certain embodiments, the S-domain is the most conserved region of the VP1 protein, which provides a rationale for the broad specificity of binding of this antibody. In additional embodiments, an exemplary family of scFv antibodies were identified that bind specifically to genogroup II-4 VLPs. These antibodies were found to function efficiently as both capture and detection reagents in ELISA experiments with GII-4 VLPs, for example. In addition, one of these exemplary antibodies, HJL-R3-B4, was shown to detect antigen from a clinical sample known to contain norovirus but not a negative control sample. Therefore, the antibodies of the present invention are useful as diagnostic agents.

### II. Antibodies of the Present Invention

Embodiments of the present invention include antibodies for the detection of Noroviruses, including but not limited to certain antibodies (for example, single chain antibodies) for identification of Norovirus or Norovirus particles. The antibodies may be of any kind, but in specific embodiments the antibodies comprise single chain antibodies (in specific embodiments, the single chain antibody comprises an antigen binding region of a light chain, an antigen binding region of a heavy chain and a flexible linker connecting the two antigen binding regions) or fragment antigen-binding (Fab fragment) antibodies, which the skilled artisan recognizes comprises one constant and one variable domain from each heavy and light chain of the antibody. In embodiments of the invention, the antibody has activity for binding with an antigen for which the antibody has specific binding affinity, and the antigen is Norovirus or Norovirus particles. Antibodies of the present invention may be generated by known methods in the art (see, for example Manual of Clinical Laboratory Immunology, Noel R. Rose, Robert G. Hamilton, Barbara Detrick (eds), American Society Microbiology; 6th edition (January 2002), 1322 pp.) or Immunology Methods Manual: The Comprehensive Sourcebook of Techniques, Ivan Lefkovits (ed.), Vol. 4, Academic Press; (Dec. 5, 1996), 2494 pp.), which are incorporated by reference herein in their entirety. For example, scFv antibodies may be generated by the methods of Miller et al. (2005), which is incorporated by reference herein in its entirety.

In some embodiments of the present invention, the antibody comprises sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and a combination thereof.

In some embodiments of the present invention, the antibody comprises 1) an amino acid segment comprising sequence selected from the group consisting of SEQ ID NO:5, SEQ ID NO:9, and SEQ ID NO:10; 2) an amino acid segment comprising sequence selected from the group consisting of SEQ ID NO:6, SEQ ID NO:11, and SEQ ID NO:12; 3) an amino acid segment comprising sequence selected from the group consisting of SEQ ID NO:7, SEQ ID NO:13, SEQ ID NO:14, SEQ ID NO:15, SEQ ID NO:16, and SEQ ID NO:17; 4) an amino acid segment comprising sequence selected from the group consisting of SEQ ID NO:8, SEQ ID NO:18, SEQ ID NO:19, SEQ ID NO:20, SEQ ID NO:21, SEQ ID NO:22, and SEQ ID NO:23; and 5) a combination thereof.

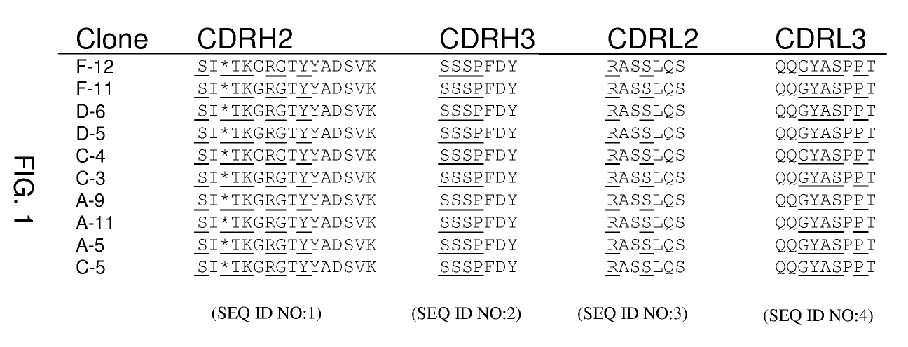

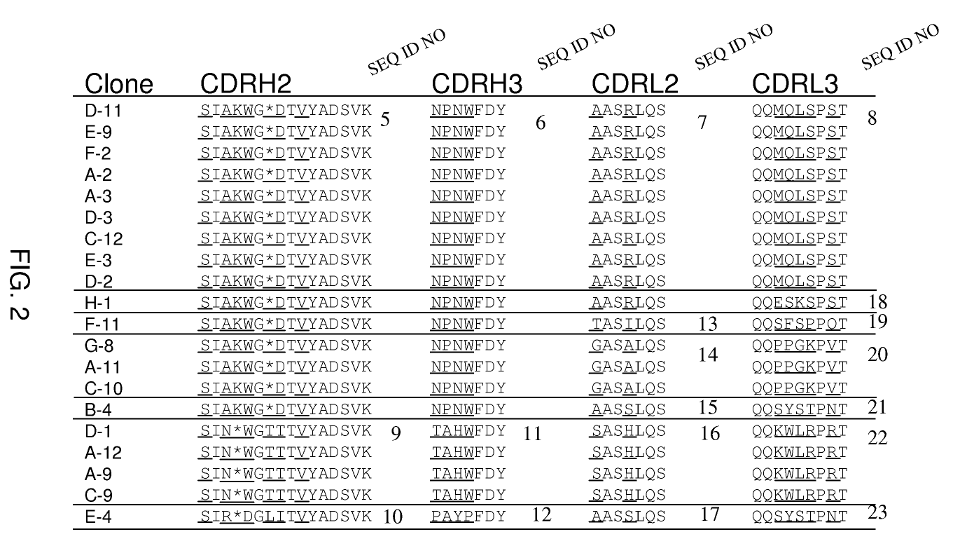

In certain cases, the sequences provided in FIGS. 1 and/or 2 are utilized in antibodies of the invention. In particular aspects, one or more variations of these sequences are employed. Although any variations of these sequences are encompassed by the invention, in specific embodiments there is variability of the sequences in one or more of the underlined regions in FIGS. 1 and/or 2. In certain embodiments, there is a peptide sequence and/or an antigen-binding region that has a peptide sequence that is 100%, 99%, 98%, 97%, 96%, 95%, 94%, 93%, 92%, 91%, 90%, 89%, 88%, 87%, 86%, 85%, 84%, 83%, 82%, 81%, or 80% to one of the sequences in FIG. 1 or 2. In particular aspects of the invention, there is a peptide sequence and/or an antigen-binding region that has a peptide sequence that is at least 99%, 98%, 97%, 96%, 95%, 94%, 93%, 92%, 91%, 90%, 89%, 88%, 87%, 86%, 85%, 84%, 83%, 82%, 81%, or 80% identical to one of the sequences in FIG. 1 or 2. In certain aspects of the invention, there is a peptide sequence and/or an antigen-binding region that has a peptide sequence that is between 80% and 85%, between 85% and 90%, between 90% and 95%, or between 95% and 99% identical to one of the sequences in FIG. 1 or 2. In certain aspects, the peptide sequence and/or an antigen-binding region is between 5 and 40 amino acids in length, between 7 and 40 amino acids in length, between 8 and 40 amino acids in length, between 9 and 40 amino acids in length, between 10 and 37 amino acids in length, between 10 and 35 amino acids in length, between 10 and 30 amino acids in length, between 10 and 25 amino acids in length, and so forth.

In particular aspects of the invention, there is a peptide sequence and/or an antibody that has a peptide sequence that has one, two, three, four, or five or more amino acid differences compared to a sequence of FIG. 1 or 2. In specific embodiments, the amino acid difference includes substitutions, deletions, and/or additions compared to a sequence of FIG. 1 or 2. In specific embodiments, there is addition, deletion, and/or substitution of one or more amino acids from the C-terminus or N-terminus of a sequence of FIG. 1 or 2. In particular aspects, there is substitution of one or more amino acids of a sequence of FIG. 1 or 2. In specific embodiments, there is a conservative substitution of one or more amino acids of a sequence of FIG. 1 or 2. In specific embodiments, one or more amino acid positions in one of the sequences of FIG. 1 or 2 is randomized, meaning that any amino acid sequence may be included therein

### III. Immunodetection Methods

In still further embodiments, the present invention concerns immunodetection methods for binding, purifying, removing, quantifying and/or otherwise generally detecting biological components such as ORF expressed message(s), protein(s), polypeptide(s), peptide(s), virions, viral-like particles, etc. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing ORF expressed message and/or protein, polypeptide, peptide, virions, and/or viral-like particles, and contacting the sample with a first anti-ORF message and/or anti-ORF translated product antibody in accordance with the present invention, as the case may be, under conditions effective to allow the formation of immunocomplexes.

These methods include methods for purifying an ORF message, protein, polypeptide and/or peptide from organelle, cell, tissue or organism's samples. In these instances, the antibody removes the antigenic ORF message, protein, polypeptide and/or peptide component from a sample. The antibody may be linked to a solid support, such as in the form of a column matrix, and the sample suspected of containing the ORF message, protein, polypeptide and/or peptide antigenic component are applied to the immobilized antibody. The unwanted components are washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody against the antigen, and then detect and quantify the amount of immune complexes formed under the specific conditions.

In terms of antigen detection, the biological sample analyzed may be any sample that is suspected of containing an antigen, such as, for example, a stool specimen, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The ORF antigen antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step labeled (such as biotinylated) monoclonal or polyclonal antibody is used to detect the target antigen(s), and a second step antibody is then used to detect the label (for example, biotin) attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various diseases wherein a specific ORF is expressed, such as an viral ORF of a viral infected cell, tissue or organism; a cancer specific gene product, etc. Here, a biological and/or clinical sample suspected of containing a specific disease associated ORF expression product is used. However, these embodiments also have applications to non-clinical samples, such as in the titering of antigen or antibody samples, for example in the selection of hybridomas.

In the clinical diagnosis and/or monitoring of patients with various forms a disease, such as, for example, cancer, the detection of a cancer specific ORF gene product, and/or an alteration in the levels of a cancer specific gene product, in comparison to the levels in a corresponding biological sample from a normal subject is indicative of a patient with cancer. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive. Of course, the antibodies of the present invention in any immunodetection or therapy known to one of ordinary skill in the art.

A. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used, for example.

In one exemplary ELISA, the antibodies of the invention are immobilized onto a selected surface (referred to as “capture” antibodies) exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of a detection antibody that is linked to a detectable label. This type of ELISA is a simple “sandwich ELISA”. In certain embodiments of the invention, the antibodies may be capture or detection antibodies.

In certain embodiments, there are variations in detection of the second antibody. In certain aspects, binding of the second antibody to the captured antigen is the detection event. The second antibody could be directly labeled with an indicator enzyme or fluorescent label or it could be detected with another antibody, for example.

Another ELISA in which the antigens are immobilized involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H2O2, in the case of peroxidase as the enzyme label. Quantification is then achieved by measuring the degree of color generated, e.g., using a visible spectra spectrophotometer.

B. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in 70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

### IV. Kits of the Invention

Any of the compositions described herein may be comprised in a kit. In a non-limiting example, one or more antibodies of the present invention may be comprised in a kit. The kits will thus comprise, in suitable container means, an antibody of the present invention. The kit may be employed for purposes of detection of Noroviruses, for example. In certain aspects, the kit is clinically available, although it may be available in institutions or settings where large groups of individuals are housed at least temporarily, for example, cruise ships, restaurants, schools, hospitals, nursing homes, day cares, and so forth.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the antibody and any other reagent containers in close confinement for commercial sale. Such containers may include injection or blow molded plastic containers into which the desired vials are retained, for example.

### EXAMPLES

The following examples are provided for further illustration of the present invention, and do not limit the invention. The examples provided herein are for illustrative purposes only, and are in no way intended to limit the scope of the present invention. While the invention has been described in detail, and with reference to specific embodiments thereof, it will be apparent to one with ordinary skill in the art that various changes and modifications can be made in the specific embodiments which are disclosed and still obtain a like or similar result without departing from the spirit and scope of the invention. Experiments and exemplary procedures are described below which provide additional enabling support for the present invention. In particular, in vitro studies using freshly isolated reactive astrocytes and in vivo studies using appropriate animal models are disclosed.

### Example 1

### Exemplary Materials and Methods

Screening of Tomlinson I+J Phage Libraries. The recombinant scFv antibody libraries were provided by MRC Geneservice. The I and J libraries are both based on a single human framework for V_{H} (V3-23/DP-47 and J_{H} 4b) and Vκ (012/02/DPK9 and Jκ1). Biopanning was initially performed with both I and J libraries. The I library has 18 residue positions CDR H2, CDR H3, CDR L2 and CDR L3 regions randomized with DVT codons (de Wildt et al., 2000). The J library has 18 residue positions in the CDR H2, CDR H3, CDR L2 and CDR L3 regions randomized with NNK codons (de Wildt et al., 2000). Both libraries are present in the pIT2 vector and consist of approximately 1.4×10⁸ independent clones.

For biopanning, Houston virus (HOV—Hu/Houston/TCH186/2002/US, Genbank EU310927) VLPs in PBS at a concentration of 5 μg/ml were added to immunotubes in a volume of 4 ml and incubated overnight at 4° C. The immunotubes were then washed three times with PBS and blocked with MPBS (2% dry milk in PBS) at room temperature for two hours followed by an additional three washes with PBS. The I and J phage libraries were then added separately to individual tubes at 10¹¹ phage in 4 ml and incubated for 2 hours at room temperature. Each immunotube was washed ten times with PBST (PBS with 0.1% Tween 20). The bound phages were eluted either by the addition of 0.5 ml of 1 mg/ml trypsin in PBS or with 0.5 ml of 5 μg/ml Le^{d} (H-type 1-PAA-biotin) carbohydrate in PBS. The elution mixtures were incubated for 10 minutes and then transferred to 1.5 ml microcentrifuge tubes. For amplification, 0.25 ml of each elution was added to 1.25 ml of E. coli TG1 cells and incubated without shaking at 37° C. for 30 minutes. Ten μl was taken, serially diluted, and spread on TYZ agar plates containing 100 μg/ml ampicillin and 1% glucose. The remaining mixture (˜1.49 ml) was spread on TYZ agar plates containing 100 μg/ml ampicillin and 1% glucose and, following overnight incubation at 37° C., the colonies were pooled. 50 μl of the pooled cells were added to 50 ml of 2YT+100 μg/ml ampicillin+1% glucose and grown at 37° C. to an OD₆₀₀ of 0.4. A total of 10 ml of this culture was incubated with 5×10¹⁰ KM13 helper phages at 37° C. for 30 min. without shaking. The culture was centrifuged at 3000 g for 10 min. and the supernatant was removed. The cell pellet was resuspended in 50 ml 2YT+100 μg/ml ampicillin+0.1% glucose+50 μg/ml kanamycin and incubated overnight with shaking at 30° C. The culture was centrifuged at 3300 g for 15 min. and the supernatant was collected. A total of 5 ml of PEG6000/2.5M NaCl was added to 20 ml of supernatant and incubated on ice for 1 hour. The mixture was centrifuged at 3300 g for 30 min. to pellet the phage particles. The phages were resuspended in 1 ml PBS, transferred to a 1.5 ml microcentrifuge tube and centrifuged at 11600 g for 10 min. to remove any remaining cells. The titer of phages in each amplification stock was determined by infecting E. coli TG1 cells. The second and third rounds of biopanning to enrich for antibody-phages that bind to HOV VLPs was performed as described above except that 20 PBST washes of bound phage were performed in round 2 and 30 washes were performed in round 3.

Single point phage ELISA. High throughput screening of phage clones was performed by single point phage ELISA (Deshayes et al., 2002). For these experiments, phages obtained after the third round of biopanning were used to infect E. coli TG1 cells and individual colonies were obtained on LB agar plates containing 100 μg/ml ampicillin and 1% glucose. Individual colonies were inoculated into 1 ml 2YT medium containing 100 μg/ml ampicillin and 1% glucose in 96-well 2 ml deep well plates and grown with shaking at 37° C. for 4 hours. A total of 10⁹ KM13 helper phage were then added to each culture well and incubated at 37° C. for 30 minutes following by centrifugation of the 96-well plate at 3000 g for 15 minutes. The supernatants were removed and the cell pellets were resuspended in 1 ml 2YT+100 μg/ml ampicillin+1% glucose and grown overnight at 30° C. The 96 well plate was centrifuged at 3000 g for 15 minutes and the supernatants were transferred to a fresh 96 well plate. For ELISA, the wells of a 96-well microtiter plate were coated with 5 μg/ml HOV or NV (Norwalk) VLPs in 100 μl total volume and incubated overnight at 4° C. The wells were washed 3 times with PBS and blocked with MPBS at room temperature for 2 hours. The wells were washed 3 times with PBS and 100 μl of each phage supernatant was added to each VLP coated well and incubated for one hour. The wells were washed 3 times with PBST (0.1% Tween 20 in PBS) and anti-M13 antibody conjugated to horseradish peroxidase was added and incubated for one hour at room temperature. The wells were washed 3 times with PBST and incubated with ABTS substrate following by absorbance data collection at OD405 in a microplate reader.

scFv phage ELISA with immobilized HOV VLPs and HOV GST-P domain fusion protein. Microplate (Immulon HB) wells were coated with 100 μl of 5 μg/ml of purified HOV VLPs, HOV GST-P domain fusion protein, GST protein or maltose binding protein and incubated overnight at 4° C. The wells were washed 3 times with 150 μl of PBS and blocked with 150 μl of MPBS for 2 hours at room temperature. The wells were washed 3 times with 150 μl of PBS and 5×1011 of each scFv display phage to be tested was added in 100 μl final volume and incubated 2 hours at room temperature. The wells were washed 3 times with 150 μl of PBS and HRP-conjugated anti-M13 antibody diluted 1:5000 in MPBS was added in 100 μl volume and incubated 45 minutes at room temperature. The wells were washed 3 times with 150 μl of PBST and 150 μl of 1-step ABTS detection reagent was added. The HRP-ABTS reaction was monitored by absorbance at OD405 in a microplate reader.

Site directed mutagenesis. The TAG amber stop codons found in the HJT-R3-A9, HJL-R3-B4, D11 and F11 scFVs were changed to CAG by site directed mutagenesis using the QuikChange Mutagenesis method (Stratagene) according to manufacturer's instructions. The HJL-R3-B4, D11 and F11 mutagenesis reactions utilized the same oligonucleotide because the CDRH2 region is identical in these clones. The DNA sequence of each scFv clone was obtained to confirm the presence of the altered codon and to ensure that extraneous mutations were not present. The primers used for mutagenesis were:

hjtr3-A9-TAG-CAG,
(SEQ ID NO: 24)
5′-GTGGGTCTCATCTATTCAGACTAAGGGTCGTGGG-3′
hjlr3-D11-TAG-CAG,
)SEQ ID NO: 25)
5′-GCTAAGTGGGGTCAGGATACAGTTTACGC-3′

scFv purification. The plasmids expressing the HJT-R3-A9, HJL-R3-B4, D11 and F11 scFVs were used to transform E. coli RB791 cells for protein expression and purification (Amann et al., 1983). The transformed cells were grown overnight at 37° C. in 10 ml of 2YT+100 μg/ml ampicillin+0.1% glucose. 10 ml of the overnight culture was used to inoculate 1 liter of 2YT+100 μg/ml ampicillin+0.1% glucose and the culture was grown at 37° C. to an OD600 of 0.8-1.0. IPTG was then added to a final concentration of 1 mM and the culture was incubated at 30° C. for five hours. The cells were harvested by centrifugation and resuspended in 50 ml of lysis buffer (25 mM sodium phosphate buffer, pH 7.4, 500 mM NaCl, 10 mM imidazole , 60 μg/ml DNAse, 1 tablet EDTA-free protease inhibitor, and 25 mM MgCl₂). A whole cell protein lysate was obtained from the resuspended cells using a French press. The resulting lysate was centrifuged for 15 minutes at 10K and the supernatant was filtered using a 0.45 μm Millipore filter. The lysate was bound with 3 ml of Talon metal affinity resin (Clontech, Inc.) incubated for 1 hour at room temperature. The resin was then packed into a column and washed with 10 bed volumes of Wash 1 buffer (25 mM sodium phosphate buffer, pH 7.4, 500 mM NaCl, 10 mM imidazole, EDTA-free protease inhibitor) and 10 bed volumes of Wash 2 buffer (25 mM sodium phosphate buffer, pH 7.4, 500 mM NaCl, 20 mM imidazole, EDTA-free protease inhibitor). Bound protein was eluted with 25 mM sodium phosphate buffer, pH 7.4, 500 mM NaCl, 50 mM imidazole, EDTA-free protease inhibitor. Elution fractions were monitored for the presence of scFv by SDS-PAGE. Fractions of high purity were pooled, concentrated, adjusted to 15% glycerol and stored at −80° C. Protein concentrations were determined using dye-binding assays (Bradford,1976).

Biacore Surface Plasmon Resonance. The binding of purified scFv to VLPs was measured using surface plasmon resonance on a Biacore 3000 instrument. For these experiments, the HOV and Norwalk (NV) VLPs were immobilized to the surface of CM-5 sensor chips. The TEM-1 β-lactamase was also immobilized to a CM-5 sensor chip as a negative control for scFv binding. The HJT-R3-A9 scFv antibody was flowed over the surface of the chip at a concentration of 16 nM to test for binding to the immobilized protein.

Immunoblotting—The HOV VLP, NV VLP, CT303 VLP and NV P-domain proteins were loaded onto an SDS-PAGE gel at a concentration of 0.3 mg/ml with a total amount of 1.5 μg loaded into each well. The proteins were fractionated by electrophoresis and transferred to nitrocellulose using a trans-blot apparatus (Bio-Rad). The membrane was blocked with 1× TBST with 5% milk at 4° C. overnight. The membrane was washed 3 times with 1× TBST with 1% milk for 10 minutes each wash. The membrane was incubated with biotinylated HJT-A9 antibody at 1.35 mg/ml in 1× TBST with 1% milk for 1 hour followed by 3 washes at 10 minutes each. Bound HJT-A9 antibody was detected with avidin-HRP (horseradish peroxidase) at 1 mg/ml in 1× TBST+1% milk for 40 minutes. The membrane was washed 3 times with 1× TBST+1% milk and 2 times with 1× TBST for 10 minutes each wash. The bands were visualized by addition of ECL reagent (Amersham) and exposure of the membrane to X-ray film.

VLP capture and detection ELISA experiments with purified scFv antibodies. Purified HJT-R3-A9, HJL-R3-B4, D11 and F11 scFv antibodies at were used to coat microplate wells (Immulon HB) with 100 μl of sample at 30 μg/ml concentration in PBS overnight at 4° C. The wells were washed 3 times with 150 μl of PBS and blocked with 150 μl of MPBS for 2 hours at room temperature. The wells were washed 3 times with 150 μl of PBS and 100 μl of 50 μg/ml of each VLP in MPBS was added to the wells and incubated for 2 hours at room temperature. Wells were washed 3 times with 150 μl of PBS and 100 μl of anti-HOV-VLP rabbit polyclonal antibody at 1:2000 dilution was added in MPBS and incubated for 1 hour at room temperature. Separate experiments were performed to show that the anti-HOV-VLP rabbit polyclonal antibody binds to the various G-II VLPs used in the experiment (data not shown). Wells were washed 3 times with 150 μl of PBS and the secondary antibody ECL anti-rabbit IgG-HRP diluted 1:1500 in MPBS was added and incubated for 1 hour at room temperature. Wells were washed 3 times with 150 μl of PBST and 150 μl of 1-step ABTS HRP substrate was added and incubated at room temperature. The ABTS reaction was monitored by absorbance at OD405 in a microplate reader.

The norovirus VLP detection assays using the mouse NS14 monoclonal antibody (Kitamoto et al., 2002) as the capture antibody were performed with 100 μl of NS14 ascites diluted 1:1000 in PBS and incubated overnight in microplate wells. The wells were washed 3 times with 150 μl of PBS and blocked with 150 μl of MPBS for 2 hours at room temperature. The wells were washed 3 times with 150 μl of PBS and 50 μg/ml of each GII VLP sample was added in 100 μl of MPBS and incubated 2 hours at room temperature. The wells were washed 3 times with 150 μl of PBS and 100 μl of 30 μg/ml HJT-R3-A9, HJL-R3-B4 or F11 antibody in MPBS was added and incubated for 1 hour at room temperature. The wells were washed 3 times with 150 μl of PBS and HRP conjugated-penta-HIS-TAG antibody diluted 1:2000 in 100 μl PBS+0.2% BSA was added and incubated at room temperature for 45 minutes. The wells were washed 3 times with PBST and 100 μl of the HRP substrate ABST was added and incubated at room temperature. The absorbance was measured at OD630 in a microplate reader.

Clinical sample ELISA using biotinylated scFV. Immulon 2 HB flat-bottom, high-binding microtiter plates (Thermo Scientific #3455) were coated with 300 ng of monoclonal antibody NS14 per well overnight at 4° C. Following washes with PBS/0.05% Tween 20, the wells were blocked with PBS/10% Blotto for 2 hours at room temperature with gentle shaking. After washing with PBS/0.05% Tween 20, a 10% stool suspension, clarified by brief centrifugation, was applied. PBS/Blotto, HOV VLP (1.6 microgram), clarified 10% virus-negative stool suspension, and clarified 10% GII.4 NV-positive stool suspension were added to duplicate wells and incubated for 2 hours at room temperature with gentle shaking. Following washing, 1 microgram/well of biotinylated scFv antibody HJL-R3-B4 in Blotto was incubated 1 hour at room temperature. After washing, a 1:5000 dilution of HRP-conjugated streptavidin (Southern Biotechnology Assoc.) in Blotto was incubated for 1 hour of incubation at room temperature. Following washes, a 1:1 mixture of TMB Peroxidase Substrate and Peroxidase Substrate Solution B (KPL) was applied and the reaction proceeded for 10 min at room temperature before addition of 1 M phosphoric acid and measurement of absorbance at 450 nm.

### Example 2

### Identification of scFv Antibodies that Bind GI and GII VLPs

In order to obtain monoclonal antibodies that bind to norovirus GI and GII VLPs, the Tomlinson I+J svFc phage display libraries (de Wildt et al., 2000) were screened for binding to Houston virus (HOV)(GII) VLPs as described in Example 1. Two methods of elution of bound phages were performed for each library. These included elution with trypsin protease, which cleaves between the scFv and the phage g3p to release the bound phages or elution by the addition of a histo-blood group antigen (HBGA) carbohydrate (H-type 1-PAA-biotin) which binds VLPs and is thought to serve as a cellular receptor for noroviruses (Hutson et al., 2003; Hutson et al., 2004). The H-type 1 carbohydrate has been shown to bind Norwalk virus VLPs and also, less efficiently, some genogroup II VLPs (Huang et al., 2005; Tan and Jiang, 2005; Choi et al., 2008). Therefore, the carbohydrate elution procedure may displace and thereby enrich for antibody-phages that bind the HOV VLPs at or near the carbohydrate receptor binding site.

After the three rounds of binding enrichment, the amplified, pooled phages from each round were tested for binding to immobilized HOV VLPs by ELISA. The highest signal was obtained with phages from the Tomlinson J libraries eluted using either trypsin or carbohydrate after three rounds of binding enrichment (data not shown). The signal from the pooled phages from the Tomlinson I library after each round of panning was significantly lower than the J library for both the trypsin and carbohydrate elutions, suggesting that relatively few phages from these enrichments bind HOV VLPs. Therefore, the remainder of the study focused on the antibody-phages enriched from the Tomlinson J library.

In order to identify individual scFv phage clones that bind to HOV VLPs, single point phage ELISA was performed using 90 randomly chosen J library clones obtained after three rounds of binding enrichment using trypsin as the elution agent (Deshayes et al., 2002). In addition, 90 J library clones obtained after three rounds of enrichment using Led carbohydrate elution were also examined (Deshayes et al., 2002). It was found that a large percentage of the clones obtained using the tryp sin elution procedure bound to HOV VLPs as indicated by a high ELISA signal compared to a negative control protein (data not shown). In order to determine if any of the antibody-phage clones also bound to a genogroup I capsid, the set of 90 clones was also tested for binding Norwalk virus (NV) VLPs by ELISA. The results indicated that a significant percentage of the clones (36/90) produced a high ELISA signal (>0.9 OD405) for both HOV and NV VLPs (data not shown). DNA sequence analysis of the scFv region of 10 of these phages revealed that they encode the same nucleotide and amino acid sequence indicating they represent a single clonal population (FIG. 1).

The single point ELISA results from the 90 clones from the carbohydrate elution revealed a number of clones that bound to HOV VLPs. The pattern of binding among clones was different than that observed for the trypsin elution. For example, although numerous clones exhibited an ELISA signal significantly above background levels, no phage clones were identified that displayed extremely high ELISA signals (>1.0 OD405) for binding HOV VLPs. In addition, the signals observed for binding to NV VLPs were, in general, low, suggesting little cross-reactivity of the scFvs between HOV and NV VLPs. DNA sequence analysis of the scFv regions of 20 phages with the highest ELISA signals for binding HOV VLPs revealed a number of different sequences that could be placed into 7 families (FIG. 2). Interestingly, several of the families possess the same heavy chain sequence. For example, 5 families encompassing 15 of the 20 sequenced clones utilize the same heavy chain sequence (FIG. 2). In addition, the 7 families are represented by 6 different light chain sequences due to the repeat of a light chain sequences in separate families. The use of a limited number of heavy chain and light chain CDR sequences that are combined in different ways to make up the 7 families suggests the antibodies bind to a similar region on the HOV VLP. Binding of the clones to a single site would be consistent with the biopanning elution procedure with carbohydrate which is designed to displace phages from the carbohydrate binding site.

### Example 3

### Characterization of scFv Antibodies that Bind Norovirus VLPs

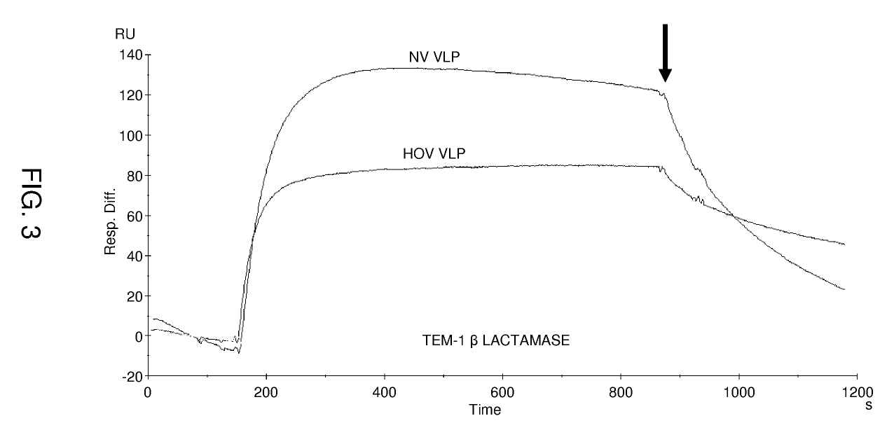

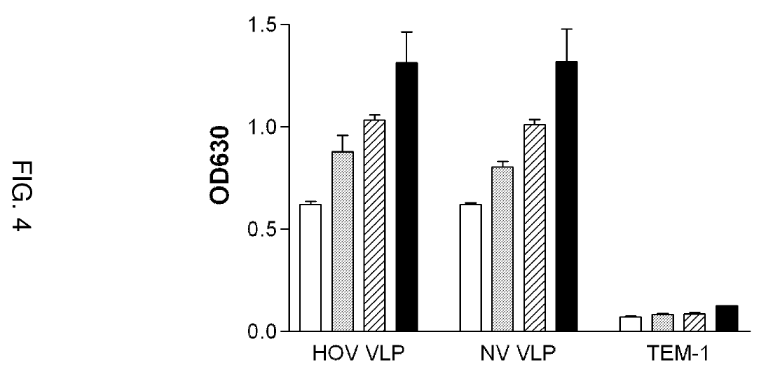

The scFv phages eluted with trypsin that bound strongly to both HOV and NV VLPs are represented by a single antibody sequence as described above. A broad spectrum scFv would be a useful diagnostic tool for norovirus infections and therefore scFv clone HJT-R3-A9 was characterized further. As indicated in FIG. 1, the CDR H2 sequence of HJT-R3-A9 contains a TAG stop codon which is suppressed to glutamine in the E. coli TG1 strain used for phage propagation. To facilitate protein expression and purification, the TAG codon was converted to the CAG glutamine codon by site directed mutagenesis. The pIT2 plasmid encoding HJT-R3-A9 was transferred to E. coli RB791 cells which do not contain a nonsense suppressor in order to express soluble scFv antibody protein. The HJT-R3-A9 protein was purified by affinity chromatography and tested for binding to HOV and NV VLPs by surface plasmon resonance (SPR) as shown in FIG. 3. The HJT-R3-A9 scFv clearly bound to HOV and NV VLPs but not to the TEM-1 β-lactamase control protein (FIG. 3). The purified HJT-R3-A9 scFv protein also bound to immobilized HOV and NV VLPs but not TEM-1 β-lactamase in ELISA experiments, which is consistent with the results of the single point phage ELISA experiments (FIG. 4). However, the inverse experiment, in which the HJT-R3-A9 antibody was immobilized in the ELISA well and soluble HOV or NV VLPs was added, washed and detected with an appropriate second antibody, did not yield an ELISA signal. This finding suggests either the HJT-R3-A9 antibody unfolds to an inactive form when coated in an ELISA well or the HOV and NV VLPs change conformation when coated in an ELISA well that reveals an epitope not available in the soluble VLPs.

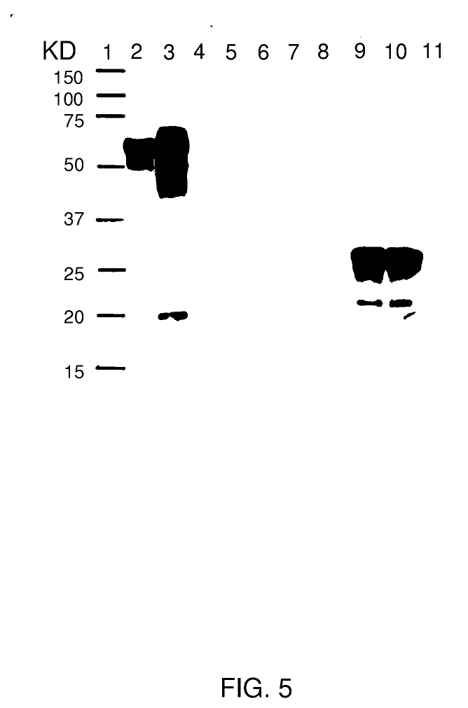

In order to gain information on the location of the HJT-R3-A9 scFv binding epitope, immunoblotting was performed. For these experiments, the HOV and NV VLPs were boiled in loading buffer and resolved by SDS-PAGE. In addition, boiled and unboiled samples of the CT303 VLP (Bertolotti-Ciarlet et al., 2002), which consists only of the S-domain, were fractionated by SDS-PAGE. Finally, a fusion protein of glutathione-S-transferase and the NV P-domain was run on SDS-PAGE with and without boiling (Parker et al., 2005). The results in FIG. 5 demonstrate that the HJT-R3-A9 antibody binds to the boiled HOV and NV VLPs. In addition, the antibody binds both the boiled and unboiled forms of the CT303 S-domain VLP but does not bind to either boiled or unboiled GST-P-domain fusion protein. Thus, the HJT-R3-A9 antibody appears to bind to a linear epitope in the S-domain. The S-domain is the most highly conserved region of VP1 between the different genogroups, which may explain the broad spectrum binding exhibited by the antibody (Chen et al., 2004). If the S-domain epitope is only exposed and available when VLPs are coated in ELISA wells but not in the soluble form, it would explain the finding that the HJT-R3-A9 scFv does not effectively capture soluble VLPs.

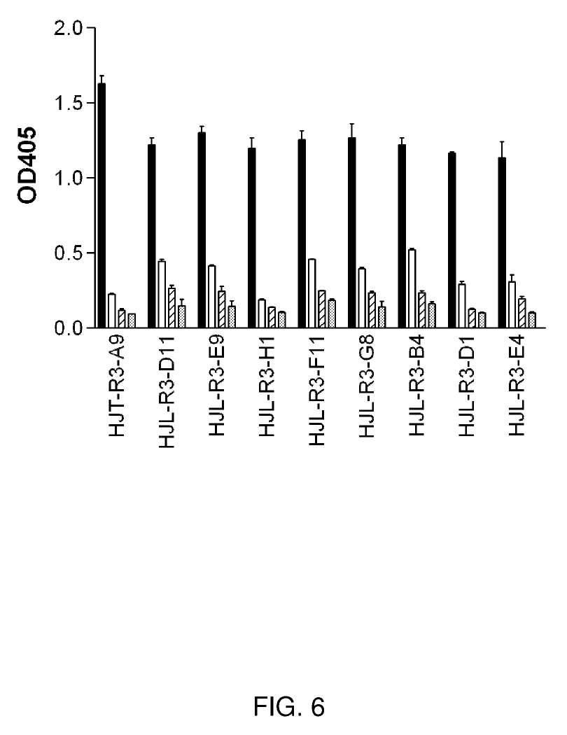

As described above, the scFv phages that were obtained from three rounds of enrichment with elution by H-type 1-PAA-biotin were found by single point phage ELISA to bind HOV but not NV VLPs. The specificity of binding of the various scFv antibodies listed in FIG. 2 was further investigated by phage ELISA. For these experiments, a representative phage clone from each scFv family in FIG. 2 as well as the HJT-R3-A9 clone from FIG. 1 was tested for binding to either immobilized HOV VLP, a GST-HOV P-domain fusion protein, GST alone, or E. coli maltose binding protein (MBP). The results in FIG. 6 reveal that each of the phage clones reacts strongly with the immobilized HOV VLP, as expected based on the single point phage ELISA experiments. In addition, the HJL-R3 clones bound to the HOV GST-P domain fusion protein, albeit with significantly lower signals than that observed for binding the HOV VLP. Also note that the HJT-R3-A9 phage bound to HOV VLP but not to the GST-P domain fusion as expected since the scFv binds to S-domain (FIG. 5). Finally, the results indicate that the scFv antibodies bind specifically to the capsid protein since the ELISA signals observed for binding of phages to GST alone or MBP were low (FIG. 6).

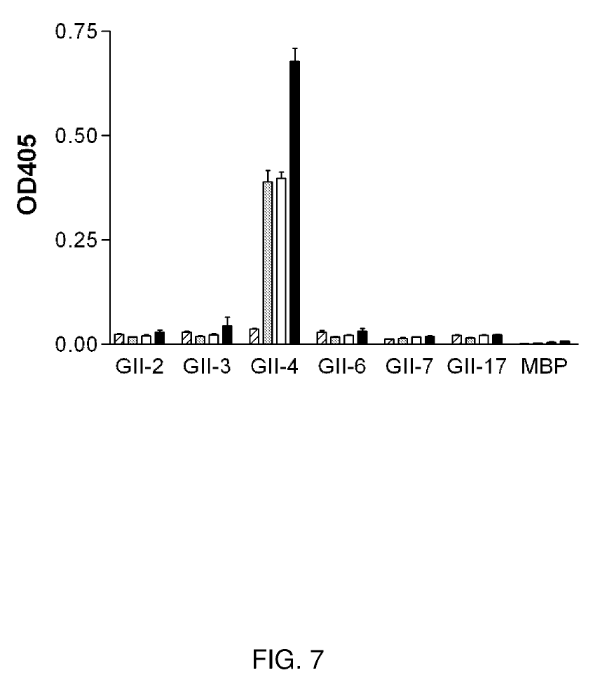

To further examine the binding characteristics of the HJL-R3 scFv antibodies, the B4, D11 and F11 scFvs (FIG. 2) were chosen for further study. The sequences in FIG. 2 indicate that each of the three types of heavy chains used among the 20 clones examined contains a TAG stop codon, as was observed for HJT-R3-A9. The TAG sequence was changed to CAG (Gln) for the HJL-R3-B4, D11 and F11 clones by site directed mutagenesis and the proteins were expressed in E. coli and purified by affinity chromatography. ELISA-based diagnostic assays for norovirus infections utilize an antibody that is present on a solid support to capture virus particles from samples followed by a second antibody to detect captured virus. Therefore, it was of interest to determine if the HJL-R3 B4, D11 and F11 antibodies could serve as capture antibodies and also to assess the specificity of capture among different GII subgroups. For this purpose, the purified HJL-R3-B4, D11 and F11 as well as the HJT-R3-A9 antibodies were coated into ELISA wells and soluble VLPs from subgroups GII-3, GII-4 (HOV), GII-6, GII-7 and GII-17 were added and allowed to bind. After washing, the bound VLPs were detected with anti-HOV rabbit polyclonal antibody. Separate experiments were performed to show that the anti-HOV-VLP rabbit polyclonal antibody binds to the various G-II VLPs (data not shown). As seen in FIG. 7, the HJL-R3-B4, D11 and F11 are all able to capture GII-4 (HOV) VLPs from solution. However, the antibodies displayed a narrow specificity in that only the HOV VLP was efficiently captured from solution. As expected based on the results described above, the HJT-R3-A9 antibody did not capture any type of the VLPs. These results suggest the HJL-R3 antibodies could be used as capture reagents in diagnostic assays to specifically identify GII-4 viruses.

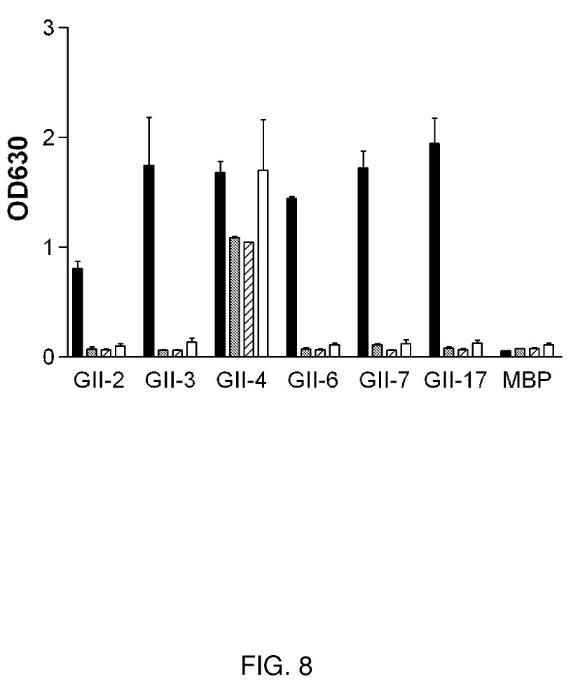

It was also of interest to determine the potential of using HJL-R3 antibodies as detection reagents in ELISA-based diagnostic assays. In addition, the HJT-R3-A9 scFv antibody does not effectively bind soluble HOV or NV VLPs indicating it would not be useful as a capture antibody in a diagnostic assay (FIG. 7). However, this antibody does detect HOV or NV VLPs coated in ELISA wells (FIG. 4). Therefore, the potential of the HJT-R3-A9 scFv as a detection antibody for diagnostics was also evaluated. For this purpose, ELISA wells were coated with the NS-14 antibody that binds GII VLPs including HOV (Kitamoto et al., 2002). Soluble VLPs from subgroups GII-3, GII-4 (HOV), GII-6, GII-7, and GII-17 were added to the NS-14 coated wells for capture. To each captured VLP was added either HJT-R3-A9 or HJL-R3-B4, D11 or F11 scFvs were added to detect the captured VLPs. After washing, the bound scFv were detected with anti-His-tag antibody. The results in FIG. 8 indicate that the HJL-R3-B4, D11 or F11 antibodies efficiently detect the captured GII-4 (HOV) VLPs but not those from other GII subgroups. This result is consistent with the specificity profile for VLP capture by these antibodies and suggests they are useful for GII-4 specific diagnostics. Interestingly, the HJT-R3-A9 antibody efficiently detected captured VLPs from all GII subtypes. This suggests that binding of these VLPs to the NS-14 capture antibody is sufficient to expose the S-domain epitope for detection.

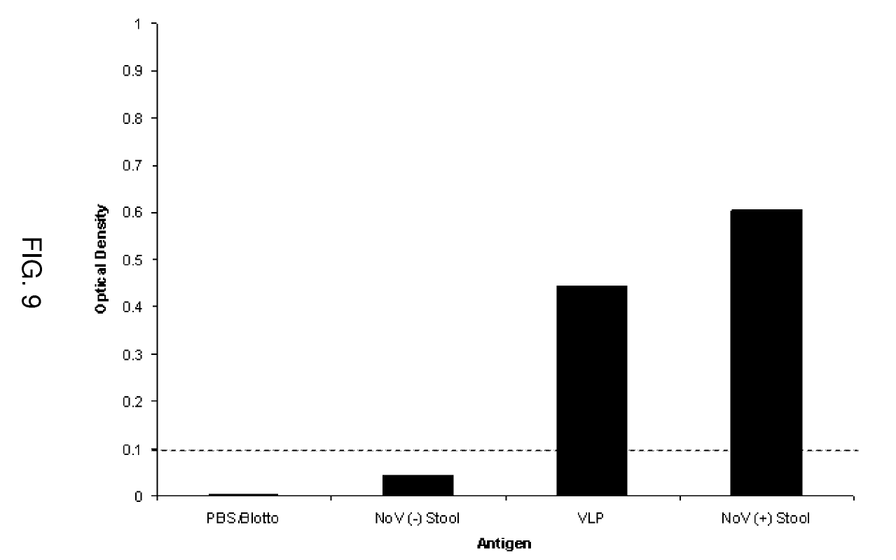

The usefulness of an antibody as a diagnostic is dependent on how efficiently it captures antigen using a clinical sample. The HJL-R3-B4 scFv was examined with stool samples that were negative for norovirus or positive for a GII.4 virus. Microtiter wells were coated with the anti-GII monoclonal antibody NS-14 as the capture antibody and stool sample suspensions were added to the wells. HOV VLPs were also used as a positive control. Purified HJL-R3-B4 antibody was labeled with biotin and added to each well, unbound protein was washed away and bound antibody was detected with streptavidin conjugated to HRP. The results in FIG. 9 show that the HJL-R3-B4 antibody bound to the HOV VLP positive control as well as the stool sample that was positive for GII.4 virus but did not bind to the stool sample negative for norovirus. Therefore, the HJL-R3-B4 antibody is able to bind and detect norovirus antigen from a clinical sample.

### Example 4

### Significance of the Present Invention

Several methods have been used to detect norovirus in diagnostic assays including electron microscopy, RT-PCR and ELISA (Atmar and Estes, 2001). The ease of use and lack of need for specialized equipment make ELISA-based methods powerful diagnostic tools. The extensive diversity of the norovirus capsid protein between and within genogroups, however, makes the development of broadly cross-reacting antibodies a challenge and currently available ELISA-based diagnostics exhibit excellent specificity but modest sensitivity because of limited cross reactivity (Richards et al., 2003; de Bruin et al., 2006). Therefore, there is a need to develop ELISA reagents with broad reactivity and high sensitivity.

In the present invention, a phage display library displaying human synthetic single chain antibodies was used to identify reagents that bind to norovirus genogroup I and II VLPs (de Wildt et al., 2000). The strategy employed was to screen the Tomlinson I+J phage libraries for antibodies that bind to GII-4 HOV VLPs and then to test the positive phage clones for binding to Norwalk GI-1 VLPs. This approach yielded multiple candidate phages but DNA sequencing indicated that these phages all encoded a single scFv sequence. A purified, soluble form of this scFv, named HJT-R3-A9 was found to efficiently detect immobilized GI and GII VLPs by ELISA. However, when the purified HJT-R3-A9 antibody was immobilized it was not effective at capturing GI or GII VLPs. Mapping of the binding site for HJT-R3-A9 by immunoblotting indicated it interacts with a linear epitope in the S-domain. This finding may explain why the antibody does not efficiently capture VLPs in that the S-domain epitope may be unavailable in VLPs in suspension in that it is in a less accessible region of VLPs than the protruding domains (Prasad et al., 1999).

A number of scFv antibodies were discovered from the phage display libraries by eluting phages bound to HOV VLPs with H type 1-carbohydrate (FIG. 2). The rationale for this approach was to specifically displace phages bound to VLPs by competition with carbohydrate so as to bias the panning experiments to enrich for scFvs that bind VLPs at or near the carbohydrate binding site. It is of interest that, although several scFvs were identified, a common heavy chain was used in 15 of the 20 clones sequenced. In addition, two light chains were found in more than one scFv (FIG. 2). Therefore, the families of scFv sequences obtained are related, which indicates the antibodies bind to a similar site on the VLP, in certain embodiments. This site corresponds to a carbohydrate binding site, in particular aspects of the invention. One could localize the binding sites for these antibodies on HOV VLPs. In contrast to the HJT-R3-A9 antibody discussed above, the antibodies identified by carbohydrate elution proved to be highly specific, not only for GII but also for only the GII-4 subgroup. This high selectivity is likely a direct result of using carbohydrate for elution in the panning protocol. It is known based on recent structural studies that histo-blood group antigens bind to different locations in different genogroups and that these regions of the capsid protein are not highly conserved (Choi et al., 2008). Therefore, the carbohydrate elution may result in the isolation of antibodies that block carbohydrate binding, but this approach, because of the varied nature of carbohydrate sites on VLPs, will lead to highly genotype-specific antibodies.

One of the goals of obtaining antibodies that bind VLPs at the carbohydrate binding site is the potential for using these reagents as prophylactics to block cell binding by noroviruses. Recently, a scFv that recognizes the carbohydrate binding site was constructed from a Mab that bound recombinant Norwalk virus VLPs (Ettayebi and Hardy, 2008). It was shown this scFv could block binding of NV VLPs to CHO cells but the binding spectrum to various genogroups was not discussed (Ettayebi and Hardy, 2008). In the present invention, several scFv were enriched from a phage library by carbohydrate elution. These scFv bind GII-4 VLPs but exhibit little cross reactivity with other GII subgroups. Nevertheless, the GII-4 strains are currently the predominant norovirus strains circulating worldwide and therefore these scFvs are useful (Fankhauser et al., 2002; Bull et al., 2006). It is noteworthy in this regard that the HJL-R3-B4 antibody is able to detect norovirus in a clinical stool sample positive for GII-4 but did not react with a negative stool sample.

### Example 5

### Clinical Embodiments of the Invention

In certain aspects of the invention, the antibodies of the present invention are employed in a clinical setting to detect Norovirus or Norovirus particles in a subject suspected of having or having been exposed to same. The skilled artisan recognizes that illness caused by norovirus infection has several names, including, for example, stomach flu, viral gastroenteritis, acute gastroenteritis, non-bacterial gastroenteritis, food poisoning, and calicivirus infection. Noroviruses reside in the stool or vomit of infected people, and people can become infected with the virus in a variety of ways, including, for example, consuming food or liquid that is contaminated with norovirus; touching surfaces contaminated with norovirus followed by placing their hand in their mouth; or having direct contact with another person who is infected and showing symptoms.

In certain cases, the individual is experiencing symptoms of Norovirus infection or has been exposed to one or more individuals experiencing same. Symptoms of Norovirus infection include nausea, abdominal pain, abdominal cramps, watery or loose diarrhea, weight loss, low-grade fever, and/or malaise, for example, although no symptoms may be apparent in some individuals that are still contagious. In certain cases, the individual is or was a hospital patient, nursing home resident, restaurant customer, catered meal eater, day care inhabitant, cruise ship passenger, student, or involved in other institutional settings, for example. The individual may be suspected to have been exposed via food-borne exposure, person-to-person exposure, or from environmental sources. In certain cases, the individual is exposed to Norovirus after consuming food and/or beverage that was contaminated. In specific cases, the invention concerns detection of Noroviruses in food and/or water for human consumption.

In specific cases, a gastrointestinal sample (such as a stool sample, for example) is obtained from an individual. The sample is diluted in buffer and there is addition to immobilized Norovirus capture antibody, either on beads or in an ELISA format, for example. The platform is washed to remove non-bound material and a second, Norovirus-specific, detection antibody is added to the immobilized sample. The sample is again washed and the second antibody is detected by methods described above, i.e., direct detection through an indicator molecule conjugated to the second antibody or via an antibody that binds to the second antibody, for example.

### REFERENCES

All patents and publications cited herein are hereby incorporated by reference in their entirety herein. Full citations for the references cited herein are provided in the following list.

### Patents

U.S. Pat. No. 3,817,837

U.S. Pat. No. 3,817,837

U.S. Pat. No. 3,850,752

U.S. Pat. No. 3,850,752

U.S. Pat. No. 3,939,350

U.S. Pat. No. 3,939,350

U.S. Pat. No. 3,996,345

U.S. Pat. No. 3,996,345

U.S. Pat. No. 4,275,149

U.S. Pat. No. 4,275,149

U.S. Pat. No. 4,277,437

U.S. Pat. No. 4,277,437

U.S. Pat. No. 4,366,241

U.S. Pat. No. 4,366,241

U.S. Pat. No. 4,472,509

U.S. Pat. No. 4,472,509

U.S. Pat. No. 4,938,948

U.S. Pat. No. 4,938,948

U.S. Pat. No. 4,938,948

U.S. Pat. No. 5,021,236

U.S. Pat. No. 5,196,066

### Publications

Amann, E., Brosius, J. and Ptashne, M. 1983 Gene 25, 167-178.

Ando, T., Monroe, S. S., Gentsch, J. R., Jin, Q., Lewis, D. C. and Glass, R. I. 1995 J. Clin. Microbiol. 33, 64-71.

Atmar, R. L. and Estes, M. K. 2001 Clin. Microbiol. Rev. 14, 15-37.

Atmar, R. L. and Estes, M. K. 2006 Gastroenterol. Clin. N. Am. 35, 275-290.

Bertolotti-Ciarlet, A., White, L. J., Chen, R., Prasad, B. V. V. and Estes, M. K. 2002 J. Virol. 76, 4044-4055.

Beuret, C., Bamumgartner, A. and Schluep, J. 2003 Appl. Environ. Microbiol. 69, 2292-2297.

Blanton, L. H., Adams, S. M., Beard, R. S., Wei, G., Bulens, S. N., Widdowson, M. A., Glass, R. I. and Monroe, S. S. 2006 J. Infect. Dis. 193, 413-421.

Bradford, M. M. 1976 Anal. Biochem. 72, 248-254.

Bu, W., Mamedova, A., Tan, M., Xia, M., Jiang, X. and Hedge, R. S. 2008 J. Virol. 82, 5340-5347.

Bull, R. A., Tu, E. T., McIver, C. J., Rawlinson, W. D. and White, P. A. 2006 J. Clin. Microbiol. 44, 327-333.

Cao, S., Lou, Z., Tan, M., Chen, Y., Liu, Y., Zhang, Z., Zhang, X. C., Jiang, X., Li, X. and Rao, Z. 2007 J. Virol. 81, 5949-5957.

Chen, R., Neill, J. D., Noel, J. S., Hutson, A. M., Glass, R. I., Estes, M. K. and Prasad, B. V. V. 2004 J. Virol. 78, 6469-6479.

Choi, J. M., Hutson, A. M., Estes, M. K. and Prasad, B. V. V. 2008 Proc. Natl. Acad. Sci. USA 105, 9175-9180.

de Bruin, E., Duizer, E., Vennema, H. and Koopmans, M. P. G. 2006 J. Virol. Methods 137, 259-264.

de Wildt, R. M. T., Mundy, C. R., Gorick, B. D. and Tomlinson, I. M. 2000 Nat. Biotechnol. 18, 989-994.

De Jager R, Abdel-Nabi H, Serafini A, Pecking A, Klein J L, Hanna M G Jr., “Current status of cancer immunodetection with radiolabeled human monoclonal antibodies” Semin Nucl Med 23(2):165-179, 1993.

Deshayes, K., Schaffer, M. L., Skelton, N. J., Nakamura, G. R., Kadkhodayan, S. and Sidhu, S. S. 2002 Chem. Biol. 9, 495-505.

Doolittle MH and Ben-Zeev O, “Immunodetection of lipoprotein lipase: antibody production, immunoprecipitation, and western blotting techniques” Methods Mol Biol., 109:215-237, 1999.

Ettayebi, K. and Hardy, M. E. 2008 Virology J. 5, 21.

Fankhauser, R. L., Monroe, S. S., Noel, J. S., Humphrey, C. D., Bresee, J. S., Parashar, U. D., Ando, T. and Glass, R. I. 2002 J. Infect. Dis. 193, 413-421.

Green, K. Y., Lew, J. F., Jiang, X., Kapikian, A. Z. and Estes, M. K. 1993 J. Clin. Microbiol. 31, 2185-2191.

Gulbis B and Galand P, “Immunodetection of the p21-ras products in human normal and preneoplastic tissues and solid tumors: a review” Hum Pathol 24(12):1271-1285, 1993.

Hansman, G. S., Natori, K., Shirato-Horikoshi, H., Ogawa, S., Oka, T., Katayama, K., Tanaka, T., Miyoshi, T., Sakae, K., Kobayashi, S., et al. 2006 J. Gen. Virol. 87, 909-919.

Hardy, M. E., Tanaka, T. N., Kitamoto, N., White, L. J., Ball, J. M., Jiang, X. and Estes, M. K. 1996 Virology 217, 252-261.

Huang, P. W., Farkas, T., Zhong, W., Tan, M., Thornton, S., Morrow, A. L. and Jiang, X. 2005 J. Virol. 79, 6714-6722.

Hutson, A. M., Atmar, R. L. and Estes, M. K. 2004 Trends in Microbiology 12, 279-287.

Hutson, A. M., Atmar, R. L., Marcus, D. M. and Estes, M. K. 2003 J. Virol. 77, 405-415.

Jiang, X., Graham, D. Y., Wang, K. N. and Estes, M. K. 1990 Science 250, 1580-1583.

Jiang, X., Wang, J. and Estes, M. K. 1995 Arch. Virol. 140, 363-374.

Jiang, X., Wang, M., Graham, D. Y. and Estes, M. K. 1992 J. Virol. 66, 6527-6532.

Jiang, X., Wilton, N., Zhong, W. M., Farkas, T., Huang, P. W., Barrett, E., Guerrero, M., Ruiz-Palacios, G., Green, K. Y., Green, J., et al. 2000 J. Infect. Dis. 181, S349-359.

Kitamoto, N., Tanaka, T., Natori, K., Takeda, N., Nakata, S., Jiang, X. and Estes, M. K. 2002 J. Clin. Microbiol. 40, 2459-2465.

Laffly, E., Pelat, T., Cedrone, F., Blesa, S., Bedouelle, H. and Thullier, P. 2008 J. Mol. Biol. 378, 1094-1103.

Lee, N., Chan, M. C., Wong, B., Choi, K. W., Sin, W., Lui, G., Chan, P. K., Cockram, C. S., Sung, J. J. and Leung, W. K. 2007 Emerg. Infect. Dis. 13, 1399-1401.

Michnick, S. W. and Sidhu, S. S. 2008 Nat. Chem. Biol. 326-329.

Miller, Keith D., Jane Weaver-Feldhaus, Sean A. Gray, Robert W. Siegel, and Michael J. Feldhaus, Production, purification, and characterization of human scFv antibodies expressed in Saccharomyces cerevisiae, Pichia pastoris, and Escherichia coli, Protein Expression and Purification, Volume 42, Issue 2, August 2005, Pages 255-267.

Parashar, U. D., and Monroe, S. S. 2001 Rev. Med. Virol. 11, 243-252.

Parker, T. D., Kitamoto, N., Tanaka, T., Hutson, A. M. and Estes, M. K. 2005 J. Virol. 79, 7402-7409.

Prasad, B. V. V., Hardy, M. E., Dokland, T., Bella, J., Rossmann, M. G. and Estes, M. K. 1999 Science 286, 287-290.

Rajpal, A., Beyaz, N., Haber, L., Cappuccilli, G., Yee, H., Bhatt, R. R., Takeuchi, T., Lerner, R. A. and Crea, R. 2005 Proc. Natl. Acad. Sci. USA 102, 8466-8471.

Richards, A. F., Lopman, B., Gunn, A., Curry, A., Ellis, D., Cotterill, H., Ratcliffe, S., Jenkins, M., Appleton, H., Gallimore, C. I. et al. 2003 J. Clin. Virol. 26, 109-115.

Sidhu, S. S. and Fellhouse, F. A. 2006 Nat. Chem. Biol. 2, 682-688.

Tan, M. and Jiang, X. 2005 Trends Microbiol. 13, 285-293.

Teunis, P. F. M., Moe, C. L., Liu, P., Miller, S. E., Lindesmith, L., Baric, R. S., Le Pendu, J. and Calderon, R. L. 2008 J. Med. Virol. 80, 1468-1476.

Tsang, O. T., Wong, A. T., Chow, C. B., Yung, R. W., Lim, W. W. and Liu, S. H. 2008 J. Hosp. Infect. 69, 135-140.

Wolf, S., Williamson, W. M., Hewitt, J., Rivera-Aban, M., Lin, S., Ball, A., Scholes, P. and Greening, G. E. 2007 Appl. Environ. Microbiol. 73, 5464-5470.

Yoda, T., Suzuki, Y., Terano, Y., Yamazaki, K., Sakon, N., Kuzuguchi, T., Oda, H. and Tsukamoto, T. 2003 J. Clin. Microbiol. 41, 2367-2371.

Zheng, D. P., Ando, T., Fankhauser, R. L., Beard, R. S., Glass, R. I. and Monroe, S. S. 2006 Virology 346, 312-323.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. An isolated peptide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

2. An isolated antibody comprising sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and a combination thereof.

3. The antibody of claim 2, further defined as a single chain antibody.

4. An antibody comprising:
1) an amino acid segment comprising sequence selected from the group consisting of SEQ ID NO:5, SEQ ID NO:9, and SEQ ID NO:10;
2) an amino acid segment comprising sequence selected from the group consisting of SEQ ID NO:6, SEQ ID NO:11, and SEQ ID NO:12;
3) an amino acid segment comprising sequence selected from the group consisting of SEQ ID NO:7, SEQ ID NO:13, SEQ ID NO:14, SEQ ID NO:15, SEQ ID NO:16, and SEQ ID NO:17;
4) an amino acid segment comprising sequence selected from the group consisting of SEQ ID NO:8, SEQ ID NO:18, SEQ ID NO:19, SEQ ID NO:20, SEQ ID NO:21, SEQ ID NO:22, and SEQ ID NO:23; and
5) a combination thereof.

5. The antibody of claim 4, further defined as a single chain antibody.

6. A kit comprising the antibody of claim 2.

7. A kit comprising the antibody of claim 4.

8. A method of detecting genogroup 11-4 Noroviruses in an individual having or suspected of having Norovirus infection, comprising the step of obtaining a sample from an individual and subjecting the sample to the antibody of claim 4.

9. The method of claim 8, wherein the antibody is labeled.

10. A method of detecting Norovirus genogroups I or II in an individual having or suspected of having Norovirus infection, comprising the step of obtaining a sample from an individual and subjecting the sample to the antibody of claim 2.

11. The method of claim 10, wherein the antibody in the subjecting step is further defined as a detection antibody in a sandwich ELISA method.

12. The method of claim 10, wherein the antibody is labeled.

13. A method of testing for Norovirus infection in an individual suspected of having a Norovirus infection or having been exposed to Norovirus, comprising the steps of:
obtaining a stool sample from the individual, wherein the individual has nausea, abdominal pain, abdominal cramps, and/or diarrhea; and
subjecting the sample to an antibody of claim 2.

14. The method of claim 13, wherein the antibody is labeled.

15. A method of testing for Norovirus infection in an individual suspected of having a Norovirus infection, comprising the steps of:
obtaining a stool sample from the individual, wherein the individual has nausea, abdominal pain, abdominal cramps, and/or diarrhea; and
subjecting the sample to an antibody of claim 4.

16. The method of claim 15, wherein the antibody is labeled.

17. An expression construct that encodes a peptide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

18. An isolated cell housing an expression construct that encodes a peptide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

19. The cell of claim 18, wherein the cell is an E. coli, yeast, mammalian, or insect cell.


SEQUENCE LISTING (25 sequences)

SEQ ID NO: 1 - protein, 16 aa, Artificial
  Synthetic peptide
SIXTKGRGTYYADSVK

SEQ ID NO: 2 - protein, 7 aa, Artificial
  Synthetic peptide
SSSPFDY

SEQ ID NO: 3 - protein, 7 aa, Artificial
  Synthetic peptide
RASSLQS

SEQ ID NO: 4 - protein, 9 aa, Artificial
  Synthetic peptide
QQGYASPPT

SEQ ID NO: 5 - protein, 16 aa, Artificial
  Synthetic peptide
SIAKWGXDTVYADSVK

SEQ ID NO: 6 - protein, 7 aa, Artificial
  Synthetic peptide
NPNWFDY

SEQ ID NO: 7 - protein, 7 aa, Artificial
  Synthetic peptide
AASRLQS

SEQ ID NO: 8 - protein, 9 aa, Artificial
  Synthetic peptide
QQMQLSPST

SEQ ID NO: 9 - protein, 16 aa, Artificial
  Synthetic peptide
SINXWGTTTVYADSVK

SEQ ID NO: 10 - protein, 16 aa, Artificial
  Synthetic peptide
SIRXDGLITVYADSVK

SEQ ID NO: 11 - protein, 7 aa, Artificial
  Synthetic peptide
TAHWFDY

SEQ ID NO: 12 - protein, 7 aa, Artificial
  Synthetic peptide
PAYPFDY

SEQ ID NO: 13 - protein, 7 aa, Artificial
  Synthetic peptide
TASILQS

SEQ ID NO: 14 - protein, 7 aa, Artificial
  Synthetic peptide
GASALQS

SEQ ID NO: 15 - protein, 7 aa, Artificial
  Synthetic peptide
AASSLQS

SEQ ID NO: 16 - protein, 7 aa, Artificial
  Synthetic peptide
SASHLQS

SEQ ID NO: 17 - protein, 7 aa, Artificial
  Synthetic peptide
AASSLQS

SEQ ID NO: 18 - protein, 9 aa, Artificial
  Synthetic peptide
QQESKSPST

SEQ ID NO: 19 - protein, 9 aa, Artificial
  Synthetic peptide
QQSFSPPQT

SEQ ID NO: 20 - protein, 9 aa, Artificial
  Synthetic peptide
QQPPGKPVT

SEQ ID NO: 21 - protein, 9 aa, Artificial
  Synthetic peptide
QQSYSTPNT

SEQ ID NO: 22 - protein, 9 aa, Artificial
  Synthetic peptide
QQKWLRPRT

SEQ ID NO: 23 - protein, 9 aa, Artificial
  Synthetic peptide
QQSYSTPNT

SEQ ID NO: 24 - DNA, 34 bp, Artificial
  Synthetic primer
gtgggtctcatctattcagactaagggtcgtggg

SEQ ID NO: 25 - DNA, 29 bp, Artificial
  Synthetic primer
gctaagtggggtcaggatacagtttacgc